(19) United States
(12) Patent Application Publication
(10) Pub. No.: US 2010/0076345 A1
(43) Pub. Date: Mar. 25, 2010

(54) METHOD, DEVICE AND SYSTEM FOR AUTOMATIC DETECTION OF EATING AND DRINKING

(76) Inventors: Edy E. Soffer, Los Angeles, CA (US); Jeffrey Conklin, West Hollywood, CA (US); Claudia Sanmiguel, Long Beach, CA (US)

Correspondence Address: BERLINER & ASSOCIATES, 555 WEST FIFTH STREET, 31ST FLOOR, LOS ANGELES, CA, 90013, US

(21) Appl. No.: 12/516,770

(22) PCT Filed: Nov. 30, 2007
(86) PCT No.: PCT/US07/86169

§ 371 Date: May 28, 2009

(51) Int. Cl. A61B 5/103

(52) U.S. Cl. 600/587

(57) ABSTRACT

A method of detecting food or drink intake in a subject by placing one or more electrodes in contact with or proximate to the subject's lower esophageal sphincter (LES), and identifying food or drink intake by monitoring electrical activity in or proximate to the LES using the one or more electrodes. The present invention also provides a device and a system for automatically detecting food or drink intake.

### CROSS REFERENCE TO RELATED APPLICATIONS

This application claims priority to U.S. Provisional Application Ser. No. 60/868,314, filed on Dec. 1, 2006, and U.S. Provisional Application Ser. No. 60/938,662, filed on May 17, 2007. Both provisional applications are incorporated herein by reference in their entireties.

### FIELD OF THE INVENTION

This invention relates generally to detection of eating and drinking, and in particular to the measurement of electrical activity indicative of eating and drinking.

### BACKGROUND

1. Related Art

Automatic detection of eating and/or drinking is a useful method that can be incorporated into a number of clinical applications in which food or drink intake is a triggering event. For example, gastric electrical stimulation (GES), by implantable devices, is a possible treatment for obesity in which electrical stimulation is applied to the stomach (Cigaina, V., Obes. Surg. 1: S12-S16, 2002; Shikora, S. A., Obes. Surg. 14: 545-548, 2004; Favretti, F., et al., Obes. Surg. 14: 666-670, 2004; Cigaina, V., Obes. Surg. 14: S14-S22, 2004). In GES, electrical signals can be delivered continuously, or, be given at the time of food intake. The latter may be advantageous when the purpose of GES is to reduce food intake. However, to deliver GES during food intake, independently of a patient's control, there is a need to automatically detect food intake and then trigger delivery of stimulation to the stomach.

There is currently one system for the detection of food intake that makes use of signals originating from electrodes sutured at different places in the stomach. The signals from the electrodes are incorporated in an algorithm that provides the automatic detection of eating (Bohdjalian, A. et al., Obes. Surg. 16: 627-634, 2004). Another system, described in U.S. Pat. No. 6,735,477, incorporated by reference herein, makes use of signals that originate from electrodes placed in the esophagus. This system measures peristaltic action in the esophagus, but does not detect electrical activity in the lower esophageal sphincter.

To fully utilize automatic eating detection in a variety of clinical situations, there exists a need in the art for alternative devices, methods and systems for the detection of food intake.

### SUMMARY

In one aspect, the present invention provides a method of detecting food or drink intake in a subject. The method comprises: a) placing one or more electrodes in contact with or proximate to the subject's lower esophageal sphincter (LES); and b) identifying food or drink intake by monitoring electrical activity in or proximate to the LES using the one or more electrodes. The pattern of electrical activity of the LES is distinct from the characteristic electrical activity pattern of peristaltic action. Thus, monitoring LES electrical activity represents a novel way to measure food and drink intake.

In some embodiments, an increase in the amplitude of the monitored electrical activity to a value greater than baseline indicates food or drink intake. In certain embodiments, a decrease in amplitude of the monitored electrical activity from a value greater than baseline to a lower value indicates cessation of food or drink intake. The electrical activity of the LES or proximate to the LES can be monitored by means of a pulse generator and a recording device, which can be separate from or a component of the pulse generator. In particular embodiments, a signal indicating food or drink intake by the subject, or indicating the cessation of food or drink intake, can be generated for use in triggering or controlling another device.

In another aspect, the present invention provides a device for automatically detecting food or drink intake of a subject. The device comprises: a) one or more electrodes, for monitoring electrical activity of the subject's LES or a region proximate to the LES; and b) a pulse generator, for generating electrical signals based on the monitored electrical activity. The device can further comprise a recording module, for recording electrical data based on the monitored electrical activity. In the device, the one or more electrodes can be functionally connected to the pulse generator or the recording module, or both,. The device can be configured to automatically identify food or drink intake by monitoring electrical activity of the LES or the region proximate to the LES using the one or more electrodes.

In a further aspect, the present invention provides a system for automatically detecting food or drink intake of a subject. The system comprises: a) one or more electrodes, for monitoring electrical activity of the subject's lower esophageal sphincter (LES) or a region proximate to the LES; b) a pulse generator, for generating electrical signals relating to the monitored electrical activity; and c) a computer for interpreting the monitored electrical activity, recording the monitored electrical activity, or both. The system can further comprise a recording device for recording electrical data relating to the monitored electrical activity. The system can be configured to automatically identify food or drink intake by monitoring electrical activity of the LES or the region proximate to the LES using the one or more electrodes.

The novel features which are believed to be characteristic of the invention, both as to its organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present invention.

### BRIEF DESCRIPTION OF DRAWINGS

FIG. 1 is a schematic drawing showing an exemplary system and method in accordance with the present invention; A pair of electrodes (vertical bars) is implanted in the lower esophageal sphincter (LES) at the level of the gastro-esophageal junction. The electrodes are connected to a pulse generator. The pulse generator is connected to a first computer, which receives data from the pulse generator and produces a signal regarding the subject's intake of food and drink. The signal is pick up by a receiver, which is connected to a second computer. All of these components can be manufactured as a single device.

FIG. 2 depicts electrode placement to record LES activity in accordance with various embodiments of the present invention. A pair of electrodes (solid bars) is placed at the junction of the esophagus and stomach.

FIG. 3 depicts recordings of LES electrical activity as a function of swallow consistency. On the y axis is electrical voltage and on the x axis is time. As shown, the amplitude of electrical activity increases transiently with swallows (*). Also, the amplitude increases as a function of what is swallowed with the highest amplitude occurring with solid food.

FIG. 4 depicts a recording of LES electrical activity as a function of swallow consistency. As shown, the amplitude of electrical activity increases as a function of what is swallowed.

FIG. 5 is a graphic depiction of the amplitude of LES electrical activity as a function of swallow consistency. The data are from several animals. As shown, the amplitude of the electrical activity increases as a function of what is swallowed, with the highest amplitude occurring with solid food.

FIG. 6 is a recording demonstrating the temporal correlation between changes in LES electrical activity and a meal. On the y axis is electrical voltage, and on the x axis is time. The onset and duration of the meal is indicated by the horizontal bar labeled “meal”. A tight temporal correlation is shown between meals and an increase in LES electrical.

FIG. 7 is a graphic depiction of the effect of a meal on LES electrical activity taken from several animals. A tight temporal correlation is shown between eating a meal and an increase in LES electrical activity.

### DETAILED DESCRIPTION

Electrical activity of the lower esophageal sphincter has been recorded mainly in vitro and in anesthetized animals. Swallowing produces changes in the motor activity of the LES. The inventors believed that these changes are related to specific changes in LES electrical activity. Thus, the inventors determined whether there are characteristic changes in LES electrical activity that can be used to recognize the beginning of a meal. Such recognition can be useful when electrical stimulation is given in conjunction with food, for example, in the treatment of obesity. Chronic studies on the use electrodes implanted in the LES to measure LES electrical activity have not heretofore been conducted. The inventors performed such a study and found that the beginning and duration of a meal can be identified by distinct, easily recognizable changes in the amplitude of LES electrical activity. These changes also depend on the type of substance being swallowed (e.g., saliva, liquid and solids), and are most prominent with solid food. Further, during fasting, transient increases in LES electrical activity not related to swallowing are associated with fundic contractions. Based on these studies, the inventors determined that changes in LES electrical activity can be used for eating detection.

The present invention provides a method of detecting food or drink intake in a subject. The method comprises: a) placing one or more electrodes in contact with or proximate to the subject's LES; and b) identifying food or drink intake by monitoring electrical activity in or proximate to the LES using the one or more electrodes.

The lower esophageal sphincter is a ring of muscle tissue located at the bottom of the esophagus where the esophagus meets the stomach. Normally, the LES acts as a valve to prevent the backflow of stomach contents into the esophagus. The junction between the esophagus and the stomach is called the gastroesophageal junction.

As used herein, “proximate” or “proximate region” to the LES generally refers to a region up to about 3 cm above or below the LES. However, one of skill in the art will appreciate that an individual's anatomy may vary and thus, an electrode can extend into a region that is more than about 3 cm above or below the LES and still be considered a proximate region as long as the change in electrical activity of the region is indicative of food or drink intake.

As used herein, “identifying food or drink intake” means identifying the start of food or drink intake, identifying ongoing food or drink intake, or identifying the cessation of food or drink intake, or any combination thereof. The term “food or drink intake” refers to food intake, drink intake, or both food intake and drink intake. Thus, in some embodiments, food intake alone is detected, while in other embodiments, drink intake alone is detected. In some embodiments, both food intake and drink intake are detected. As used herein, “food intake” means the intake of solid food, and “drink intake” means the intake of a liquid.

“About” a certain value (i.e. about 0.43 mV) means within the experimental error typical of such measurements.

In one embodiment, one or more electrodes are placed in contact with the LES or in contact with a proximate region to the LES and the electrical activity is monitored at that location. An increase in the amplitude of electrical activity in the monitored location indicates food or drink intake, and a decrease in the amplitude back to about baseline level indicates the cessation of food or drink intake. Further, the duration of the change in amplitude (e.g., increase in amplitude) can be used to differentiate between types of swallows. For example, a short duration indicates simple swallows or a very small snack and a long duration indicates the consumption of a meal. The electrical activity of the LES while in a resting or non-swallowing state can establish the baseline level, and amplitudes above the baseline can indicate dry swallows, wet swallows, or solid food swallows, depending on the size and duration of the amplitudes.

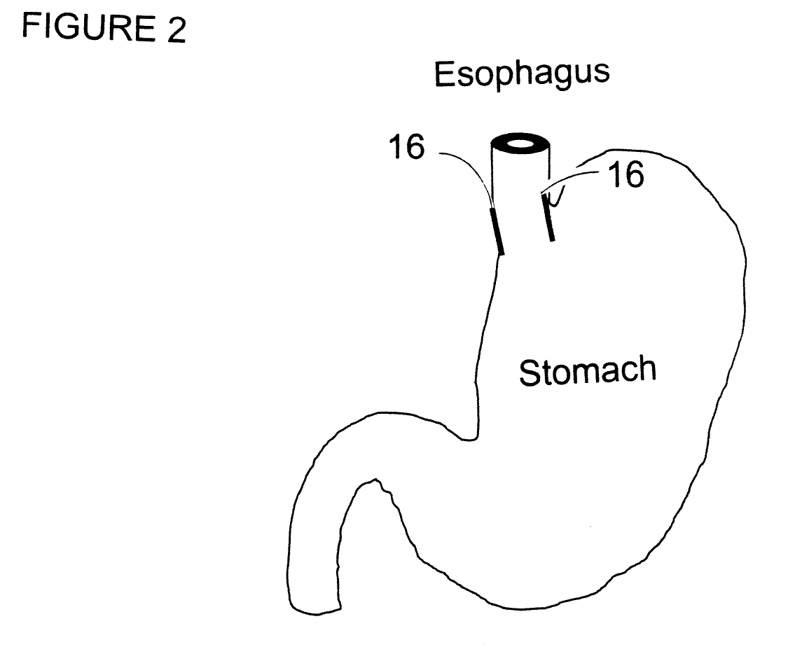

In some embodiments, a pair of electrodes is placed. As shown in FIG. 2, two electrodes 16 can be positioned at opposite sides of the gastroesophageal junction (GEJ). In particular embodiments, one electrode is positioned in the anterior aspect of the GEJ and a second electrode is positioned in the posterior aspect of the GEJ.

In other embodiments, one or more electrodes are positioned away from the vagus nerve trunks. In a particular embodiment, one or more electrodes are positioned as far away from the vagus nerve trunks as possible so long as electrical activity indicative of food or drink intake can be detected. In a particular embodiment, two electrodes are positioned as far away from the vagus nerve trunks as possible.

An electrode can be of any size suitable for placement on or in the LES, or on or in a proximate region to the LES. In various embodiments, the electrodes can be about 1 mm long to about 50 mm long, about 5 mm long to about 25 mm long, or about 10 mm long to about 20 mm long. In one embodiment, the electrode can be about 15 mm long. The electrode can also be of any shape suitable for placement on the LES or on a proximate region to the LES; for example, circular, square, rectangular, etc. The electrode can also be of any dimension suitable for placement on the LES or on a proximate region to the LES. The electrode can be attached on the surface of the LES or proximate region, or implanted into the LES or proximate region.

Placing an electrode in contact with the LES or proximate to the LES can be performed by any method known in the art; for example, by a surgical procedure or by an endoscopic procedure. The electrode can be placed on any level in the LES tissue from the inner lining (i.e., mucosa) to the muscle layer. In one particular embodiment, an electrode can be sutured to a muscle layer of the LES or a proximate region to the LES.

In some embodiments, monitoring the electrical activity comprises detecting the electrical activity in the LES. In particular embodiments, monitoring the electrical activity comprises measuring the amplitude and/or duration of the electrical activity in the LES.

In accordance with the present invention, an increase in amplitude of the monitored electrical activity to a value greater than baseline amplitude can indicate food or drink intake. In certain embodiments, an about three to about four fold increase in amplitude from baseline amplitude indicates food or drink intake.

In some embodiments, an amplitude of about 0.30 mV to about 0.90 mV indicates a dry swallow, or an amplitude of about 0.40 mV to about 0.80 mV, about 0.45 mV to about 0.75 mV, or about 0.5 mV to about 0.7 mV indicates a dry swallow. In a particular embodiment, an amplitude of about 0.6 mV indicates a dry swallow. Alternatively, an about two-fold increase in amplitude indicates a dry swallow. A “dry swallow” is a swallow in the absence of food or drink.

In some embodiments, an amplitude of about 0.31 mV to about 1.03 mV indicates a drink intake (wet swallow), or an amplitude of about 0.43 mV to about 0.91 mV, about 0.52 mV to about 0.88 mV, or about 0.58 mV to about 0.82 mV indicates a drink intake. In a certain embodiment, an amplitude of about 0.7 mV indicates a drink intake. Alternatively, an about two-fold increase in amplitude indicates a wet swallow.

In some embodiments, an amplitude of about 0.55 mV to about 1.57 mV indicates solid food intake, or an amplitude of about 0.72 mV to about 1.4 mV, about 0.81 mV to about 1.32 mV, or about 0.89 mV to about 1.23 mV indicates solid food intake. In a particular embodiment, an about 1.06 mV indicates solid food intake. Alternatively, a greater than three-fold increase in amplitude indicates solid food intake, or an about three to about four fold increase in amplitude indicates solid food intake.

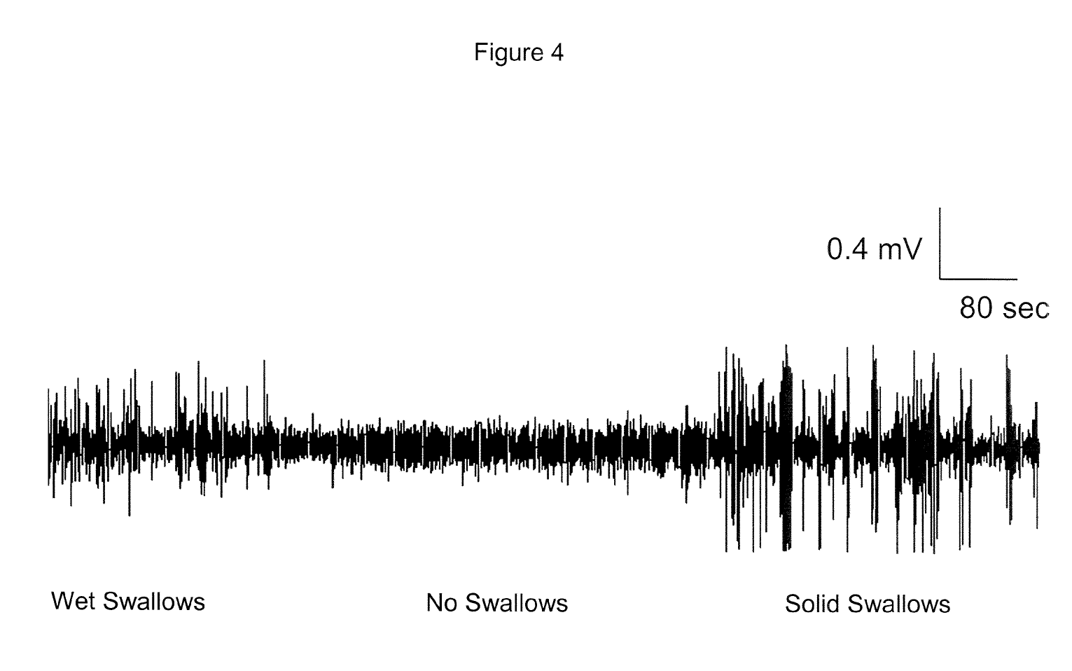

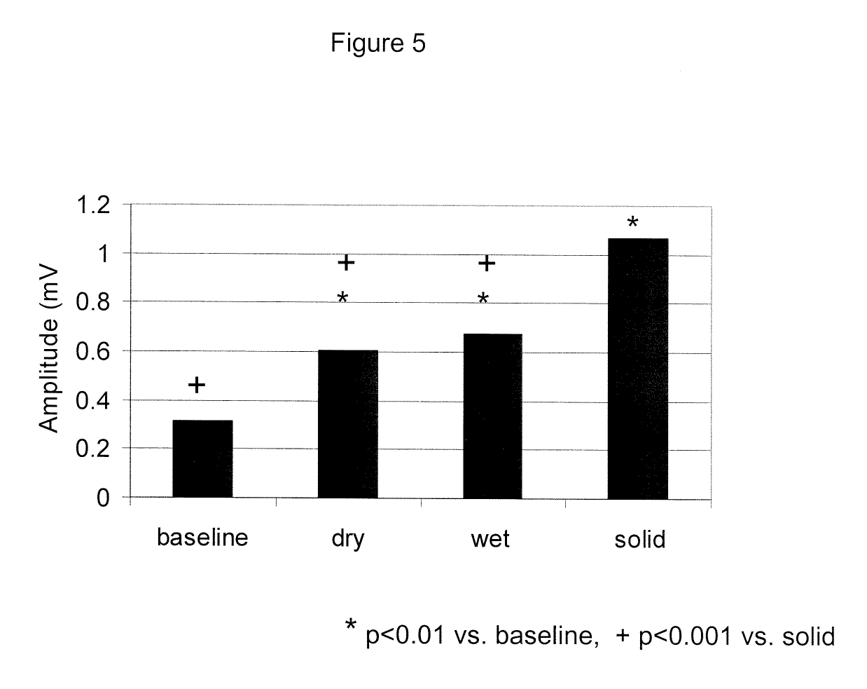

The specific amplitudes indicative of dry swallows, wet swallows and food intake will vary depending on the subject being examined. The range of amplitudes for a specific subject can be obtained by measuring the subject's background level of electrical activity while the subject is in a resting or non-swallowing state, then measuring the amplitudes when the subject is performing a dry swallow, is swallowing liquid, and is swallowing solid food. These observed amplitudes can be used to identify background electrical activity and different types of swallows when the subject is subsequently monitored for food or drink intake (see FIGS. 4 and 5).

In accordance with the present invention, reversion of an increased amplitude back to baseline or to a value of approximately baseline amplitude can indicate that food or drink intake has stopped. Further, a decrease in amplitude from a higher value to a lower value can indicate that food or drink intake has stopped. In some embodiments, an about three to about four fold decrease in amplitude from the increased amplitude indicates food or drink intake has stopped. In certain embodiments, an amplitude of about 0.135 mV to about 0.495 mV indicates that food or drink intake has stopped, or an amplitude of about 0.195 mV to about 0.435 mV, about 0.225 mV to about 0.405 mV, or about 0.255 mV to about 0.375 mV indicates that food or drink intake has stopped. In a particular embodiment, an amplitude of about 0.315 mV indicates that food or drink intake has stopped.

To record data, an electrode can be connected to a recording module. The recording module can be incorporated as part of a pulse generator to measure the electrical activity in the LES. In other embodiments, a recording device can be a separate component from the pulse generator. In certain embodiments, the pulse generator is implantable. In certain embodiments, the recording device is an implantable recording device. Connections between the pulse generator and the recording device can be by wired connection, or by wireless connection. In preferred embodiments, a recording of the electrical activity is obtained by placing wands on the subject's skin that detect the electrical activity, and connecting the wands to data loggers.

In some embodiments, a signal indicating that a subject has started consuming food or drink, is in the process of consuming food or drink, has stopped consuming food or drink, has consumed food or drink, or any combination thereof, can be generated based on the amplitude of the electrical activity of the LES or proximate to the LES. The signal can be sent to a receiving device, such as a computer or a system containing a receiving device, or other device or system associated with food or drink intake or the cessation of food or drink intake. As such, additional embodiments of the present invention can further comprise using a receiver to receive signals regarding the subject's food or drink intake. In preferred embodiments, the receiving device is used in a clinical application associated with food or drink intake. Thus, the detection of food or drink intake or cessation of food or drink intake, or signals indicative thereof, may be used in conjunction with other technology for clinical applications. That is, the detection of food or drink intake or the cessation of food or drink intake, or signals indicative thereof, can be used to trigger an intervention treatment that is associated with the food or drink intake or the cessation of food or drink intake. Examples of such intervention include but are not limited to technology that induces changes in the motor activity of the stomach that can affect food intake, technology that delivers a stimulus in conjunction with food intake to affect food intake, technology that applies gastric electrical stimulation to induce satiety, technology that treats obesity, technology that treats diabetes (e.g., insulin pumps), technology that administers biochemical agents that affect eating, technology that modifies esophageal and/or LES motility, and technology that stimulates the esophagus or the LES.

Additional embodiments of the present invention can further comprise using a computer or computer system to perform a number of functions, for example, including but not limited to receiving electrical signals, analyzing electrical signals, processing electrical signals, and sending a signal regarding the received, analyzed and/or processed electrical signals to another system, computer or device. Such computers and computer systems are known in the art and one of skill in the art will be able to determine, without undue experimentation, a computer or a computer system that is suitable for such use.

The present invention also provides a device for practicing the method of detecting food or drink intake of the present invention. The device, or “detection device”, comprises: a) one or more electrodes, for monitoring electrical activity of the subject's LES or a region proximate to the LES; and b) a pulse generator, for generating electrical signals based on the monitored electrical activity. The device can further comprise a recording module, for recording electrical data based on the monitored electrical activity. The one or more electrodes can be functionally connected to the pulse generator or the recording module, or both, and the device can be configured to identify food or drink intake by monitoring electrical activity of or proximate to the LES using the one or more electrodes. In some embodiments, one or more pairs of electrodes is utilized. The detection device can be configured to automatically detect food or drink intake in a subject.

In one embodiment, the detection device comprises one or more electrodes, a pulse generator and a recording module, wherein the one or more electrodes is connected to the pulse generator or the recording device, or both. The one or more electrodes can be one or more pairs of electrodes. The device is configured to detect food or drink intake by measuring the electrical activity in the LES or in the proximate region to the LES. The pulse generator can be used to generate pulses or signals that are read and processed by a computer or recorded by the recording unit, or both. In particular embodiments, the detection device is configured to measure the amplitude and/or duration of the electrical activity in the LES or in the proximate region to the LES. In some embodiments, the detection device is an implantable device.

The recording device can be incorporated as part of the pulse generator, or can be a separate component from the pulse generator. In certain embodiments, the pulse generator is implantable. In certain embodiments, the recording device is an implantable recording device. Connections between the pulse generator and the recording device can be by wired connection, or by wireless connection. In preferred embodiments, a recording of the electrical activity is obtained by placing wands on the subject's skin that detect the electrical activity, and connecting the wands to data loggers.

The electrode can be any size suitable for placement on the LES or a proximate region to the LES. In various embodiments, the electrodes can be about 1 mm long to about 50 mm long, about 5 mm long to about 25 mm long, or about 10 mm long to about 20 mm long. In one embodiment, the electrode may be about 15 mm long. The electrode can be any shape suitable for placement at the LES; for example, circular, square, rectangular, etc., and can be any dimension suitable for placement at the LES.

In some embodiments, the detection device can further comprise a computer. The computer can be used to perform a number of functions; for example, including but not limited to receiving electrical signals, analyzing electrical signals, processing electrical signals, and sending a signal regarding the received, analyzed and/or processed electrical signals to another system, computer or device.

In one embodiment, the detection device is configured to generate and send a signal to another device indicating the electrical activity of the LES. In some embodiments, the signal can be a signal that indicates that the subject has started consuming food or drink, is consuming food or drink, has stopped consuming food or drink, has consumed food or drink, or any combination thereof.

In some embodiments, the detection device is configured to generate and send a signal when an increase in amplitude from baseline amplitude is detected. In another embodiment, the device is configured to generate and send a signal that the subject has consumed food or drink when an about three to about four fold increase in amplitude from a baseline amplitude is detected.

In some embodiments, the detection device is configured to generate and send a signal that the subject has swallowed when an amplitude of about 0.30 mV to about 0.90 mV has been detected. In particular embodiments, the device is configured to generate and send a signal that the subject has swallowed when an amplitude of 0.40 mV to about 0.80 mV, about 0.45 mV to about 0.75 mV, or about 0.5 mV to about 0.7 mV has been detected. In a particular embodiment, the device is configured to generate and send a signal that the subject has swallowed when an amplitude of about 0.6 mV has been detected. Alternatively, the device is configured to generate and send a signal that the subject has swallowed when an about two-fold increase in amplitude has been detected.

In some embodiments, the detection device is configured to generate and send a signal that the subject has consumed a liquid when an amplitude of about 0.31 mV to about 1.03 mV, about 0.43 mV to about 0.91 mV, about 0.52 mV to about 0.88 mV, or about 0.58 mV to about 0.82 mV has been detected. In a particular embodiment, the device is configured to generate and send a signal that the subject has consumed a liquid when an amplitude of about 0.7 mV has been detected. Alternatively, the device is configured to generate and send a signal that the subject has consumed a liquid when an about two-fold increase in amplitude has been detected.

In some embodiments, the detection device is configured to generate and send a signal that the subject has consumed solid food when an amplitude of 0.55 mV to about 1.57 mV has been detected. In certain embodiments, the device is configured to generate and send a signal that the subject has consumed food when amplitude of about 0.72 mV to about 1.4 mV, about 0.81 mV to about 1.32 mV, or about 0.89 mV to about 1.23 mV has been detected. In a particular embodiment, the device is configured to generate and send a signal that the subject has consumed food when amplitude of about 1.06 mV has been detected. Alternatively, the device is configured to generate and send a signal that the subject has consumed food when greater than a two-fold increase in amplitude has been detected. In particular embodiments, the device is configured to generate and send a signal that the subject has consumed food when an about three to about four fold increase in amplitude is detected.

In accordance with the present invention, reversion of an increased amplitude back to baseline or to a value of approximately baseline amplitude can indicate that food or drink intake has stopped. Further, a decrease in amplitude from a higher value to a lower value can indicate that food or drink intake has stopped. In some embodiments, the detection device is configured to generate and send a signal that the subject has ceased consuming food or drink when a reversion of the increased amplitude back to approximately baseline amplitude is detected. In certain embodiments, the device is configured to generate and send a signal that the subject has stopped consuming food or drink when an about three to about four fold decrease in amplitude from the increased amplitude is detected.

In some embodiments, the detection device can be configured to generate and send a signal that the subject has ceased consuming food or drink when an amplitude of about 0.135 mV to about 0.495 mV has been detected. In particular embodiments, the device may be configured to generate and send a signal that the subject has ceased consuming food or drink when amplitude of about 0.195 mV to about 0.435 mV, about 0.225 mV to about 0.405 mV, about 0.255 mV to about 0.375 mV has been detected. In a particular embodiment, the device is configured to generate and send a signal that the subject has ceased consuming food when an amplitude of about 0.315 mV has been detected.

These signals may be useful for a variety of clinical applications. The signals may be used in conjunction with other technology for clinical applications. That is, the signal generated when food or drink intake is detected or when the cessation of food or drink intake is detected may be used to trigger an intervention treatment that is associated with the food or drink intake or the cessation of food or drink intake. Examples of such intervention include but are not limited to technology that induces changes in the motor activity of the stomach that can affect food intake, technology that delivers a stimulus in conjunction with food intake to affect food intake, technology that applies gastric electrical stimulation to induce satiety, technology that treats obesity, technology that treats diabetes (e.g., insulin pumps), technology that administers biochemical agents that affect eating, technology that modifies esophageal and/or LES motility, and technology that stimulates the esophagus or the LES.

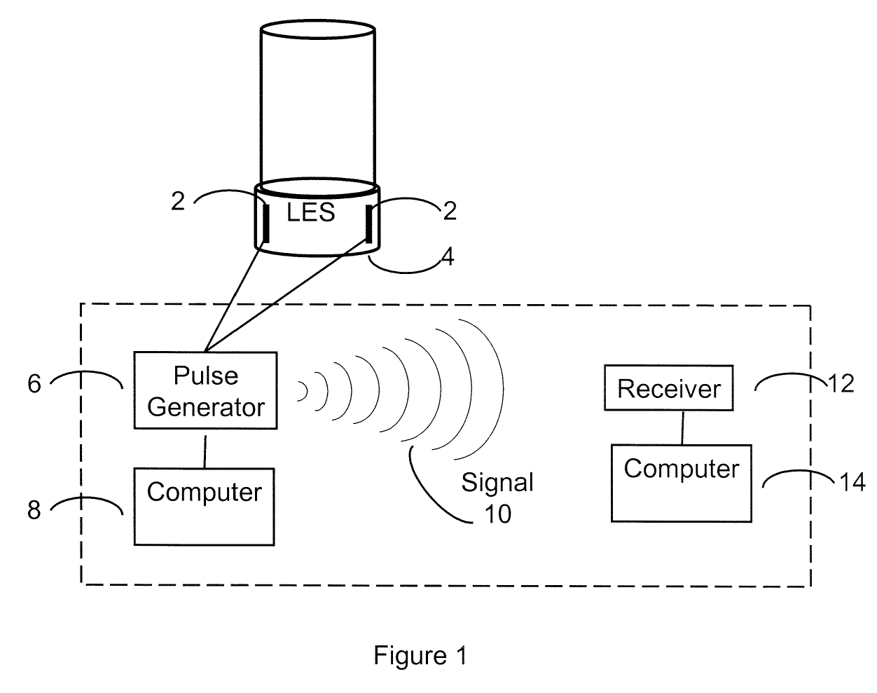

The present invention also provides a system for practicing the method of the present invention. A schematic drawing of an embodiment of the system is shown in FIG. 1. A pair of electrodes 2 (vertical bars) is implanted in the lower esophageal sphincter 4 (LES) at the level of the gastro-esophageal junction. The electrodes are connected to a pulse generator 6. The pulse generator is connected to computer 8, which receives data from the pulse generator and produces a signal 10 regarding the subject's intake of food and drink. The signal is pick up by a receiver 12, which is connected to computer 14. All of these components can be manufactured as a single device. In some embodiments, computer 14 can be a gastric electrical stimulation device for obesity treatment. The system can be configured to automatically detect food or drink intake.

In one embodiment, the system comprises a device for monitoring the electrical activity of the LES and a computer for interpreting and/or recording the electrical activity of the LES. In another embodiment, the system further comprises a device for recording the electrical activity of the LES. The device for monitoring the electrical activity can comprise one or more electrodes, a pulse generator, and a recording module, wherein the pulse generator or the recording module, or both, can be connected to the one or more electrodes and the device is configured to measure the electrical activity in the LES or in a proximate region to the LES. In particular embodiments, the device for monitoring the electrical activity is configured to measure the amplitude and/or duration of the electrical activity in the LES or in the proximate region to the LES. The pulse generator can be used to generate pulses or signals that are read and processed by a computer.

The electrode can be any size suitable for placement at the LES. In various embodiments, the electrode can be about 1 mm long to about 50 mm long, about 5 mm long to about 25 mm long, or about 10 mm long to about 20 mm long. In one embodiment, the electrode is about 15 mm long. The electrode can be any shape suitable for placement at the LES, such as circular, square, rectangular, etc. The electrode can also be of any dimension suitable for placement at the LES.

A computer can be used to perform a number of functions, for example, including but not limited to receiving electrical signals, analyzing electrical signals, processing electrical signals, and sending a signal regarding the electrical signals to another system, computer or device

Additional embodiments of the system further comprise a receiver for receiving signals regarding a subject's food or drink intake.

The system can comprise a device for monitoring the electrical activity and a device for sending a signal to a second system or device. In one embodiment, the second system or device is a system or device for the treatment of obesity.

The device for sending a signal to a second system or device can be configured to generate and send a signal to indicate the electrical activity of the LES. In particular embodiments, the signal is a signal indicating that the subject has started consuming food or drink, is in the process of consuming food or drink, has stopped consuming food or drink, has consumed food or drink, or any combination thereof.

In some embodiments, the device for sending a signal is configured to generate and send a signal when an increase in amplitude from baseline amplitude is detected. In certain embodiments, the device is configured to generate and send a signal that the subject has consumed food or drink when an about three to about four fold increase in amplitude from a baseline amplitude is detected.

In some embodiments, the device is configured to generate and send a signal that the subject has swallowed when an amplitude of about 0.30 mV to about 0.90 mV has been detected. In particular embodiments, the device is configured to generate and send a signal that the subject has swallowed when an amplitude of 0.40 mV to about 0.80 mV, about 0.45 mV to about 0.75 mV, or about 0.5 mV to about 0.7 mV has been detected. In a particular embodiment, the device is configured to generate and send a signal that the subject has swallowed when an amplitude of about 0.6 mV has been detected. Alternatively, the device is configured to generate and send a signal that the subject has swallowed when an about two-fold increase in amplitude has been detected.

In some embodiments, the device is configured to generate and send a signal that the subject has consumed a liquid when amplitude of about 0.31 mV to about 1.03 mV has been detected. In other embodiments, the device is configured to generate and send a signal that the subject has consumed a liquid when amplitude of 0.43 mV to about 0.91 mV, about 0.52 mV to about 0.88 mV, or about 0.58 mV to about 0.82 mV has been detected. In a particular embodiment, the device is configured to generate and send a signal that the subject has consumed a liquid when amplitude of about 0.7 mV has been detected. Alternatively, the device is configured to generate and send a signal that the subject has consumed a liquid when an about two-fold increase in amplitude has been detected.

In some embodiment, the device is configured to generate and send a signal that the subject has consumed food when an amplitude of 0.55 mV to about 1.57 mV has been detected. In other embodiments, the device is configured to generate and send a signal that the subject has consumed food when an amplitude of about 0.72 mV to about 1.4 mV, about 0.81 mV to about 1.32 mV, or about 0.89 mV to about 1.23 mV has been detected. In a particular embodiment, the device is configured to generate and send a signal that the subject has consumed food when an amplitude of about 1.06 mV has been detected. Alternatively, the device is configured to generate and send a signal that the subject has consumed food when greater than a two-fold increase in amplitude has been detected. In particular embodiments, the device is configured to generate and send a signal that the subject has consumed food when an about three to about four fold increase in amplitude is detected.

In accordance with the present invention, reversion of an increased amplitude back to baseline or to a value of approximately baseline amplitude can indicate that food or drink intake has stopped. Further, a decrease in amplitude from a higher value to a lower value can indicate that food or drink intake has stopped. In some embodiments, the device is configured to generate and send a signal that the subject has ceased consuming food or drink when a reversion of the increased amplitude back to approximately baseline amplitude is detected. In particular embodiments, the device is configured to generate and send a signal that the subject has stopped consuming food or drink when an about three to about four fold decrease in amplitude from the increased amplitude is detected.

In some embodiment, the device is configured to generate and send a signal that the subject has ceased consuming food or drink when an amplitude of about 0.135 mV to about 0.495 mV has been detected. In certain embodiments, the device is configured to generate and send a signal that the subject has ceased consuming food or drink when an amplitude of about 0.195 mV to about 0.435 mV, about 0.225 mV to about 0.405 mV, about 0.255 mV to about 0.375 mV has been detected. In a particular embodiment, the device is configured to generate and send a signal that the subject has ceased consuming food when an amplitude of about 0.315 mV has been detected

The signal sent by the signal sending device can be used by a second device or system. The second device or system can be a technology for a clinical application. That is, the second device or system can be an intervention treatment that is associated with food or drink intake or the cessation of food or drink intake. Examples of such intervention include but are not limited to technology that induces changes in the motor activity of the stomach that can affect food intake, technology that delivers a stimulus in conjunction with food intake to affect food intake, technology that applies gastric electrical stimulation to induce satiety, technology that treats obesity, technology that treats diabetes (e.g., insulin pumps), technology that administers biochemical agents that affect eating, technology that modifies esophageal and/or LES motility, and technology that stimulates the esophagus or the LES.

In a particular embodiment, the second device is a gastric stimulation device to treat obesity. For example, the signal can prompt the gastric stimulation device to start gastric stimulation to induce safety and/or prompt the gastric stimulation device to cease gastric stimulation.

The present invention may be better understood by referring to the accompanying examples, which are intended for illustration purposes only and should not in any sense be construed as limiting the scope of the invention as defined in the claims appended hereto.

### Example 1

### Implantation Technique and Recordings

Two electrodes were implanted in the muscularis of the lower esophageal sphincter (LES) and fundus of four female mongrel dogs (23.1±2.3 kg). (See FIG. 2.) The electrodes were 15 mm long, and were sutured to the muscle layer of the LES, at the level of the gastro-esophageal junction. The electrodes were connected to a stimulation/recording device. Particularly, the electrodes were connected to an implantable pulse generator that was positioned under the skin of the flank of the dogs. Accurate positioning was verified by endoscopy done at the time of operation. The two electrodes were positioned at opposite sides of the gastro-esophageal junction (GEJ). In a dog, the distance between them is about 2 cm. One electrode was positioned in the anterior aspect of the GEJ, and the second one in the posterior aspect of the GEJ, essentially as far away from the vagus nerve trunks as possible. A cervical esophagostomy was performed to facilitate esophageal manometry. Recordings of the LES electrical activity and fundic mechanical activity (impedance) were recorded by telemetry. Special wands were placed on the skin and connected to data loggers. In this way, recordings of electrical activity could be obtained from dogs that were able to move in their cages freely and to eat freely. A button on the recorder was used to mark the beginning and end of the meal.

### Example 2

Study design

After recovery, dogs were fitted with the wands and recorders in their cages. Recordings were obtained under light sedation (acepromazine: 0.5 mg/kg). Manometry was performed with water-perfused or solid-state manometer systems. Continuous recordings were obtained for: baseline, dry swallows, wet swallows (10 cc water/each) and solid food (can food, 400 g).

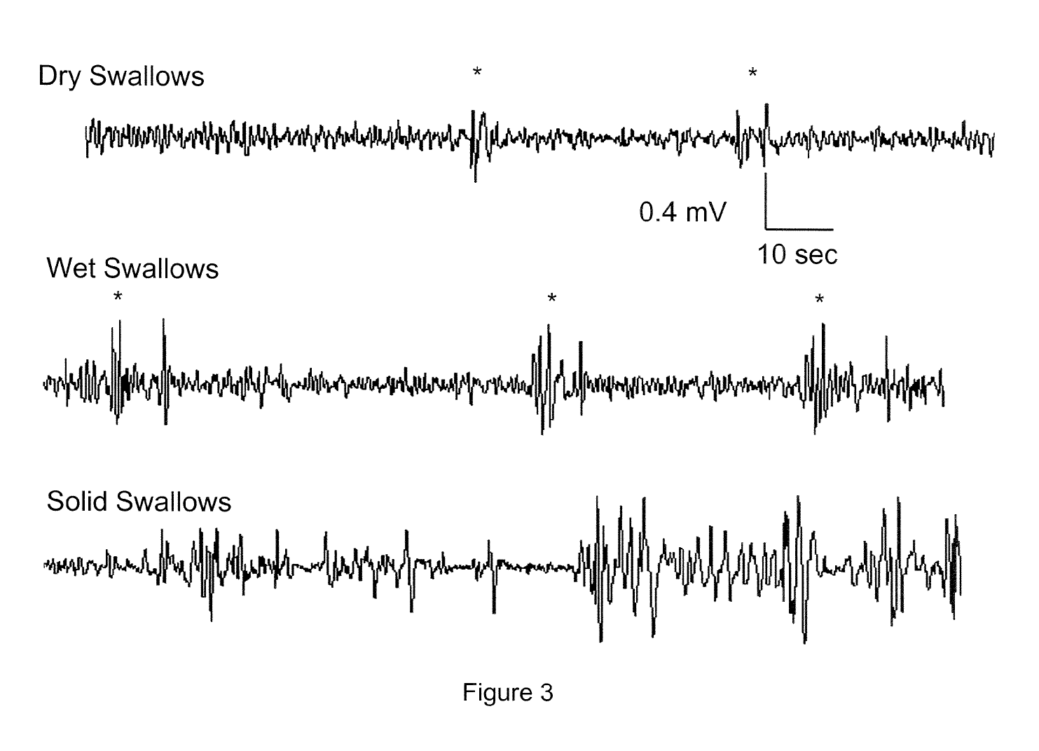

Continuous recordings of LES electrical activity were recorded by telemetry for the following periods: dry swallows, wet swallows, and the beginning and end of solid meals (FIG. 3).

The amplitude of LES electrical activity was determined for 2 minutes during the baseline, and during dry, wet and solid food swallows. A statistical test, analysis of variance (ANOVA) was used to determine if there was a statistical differences in amplitude of LES electrical signals between periods (FIG. 5).

### Example 3

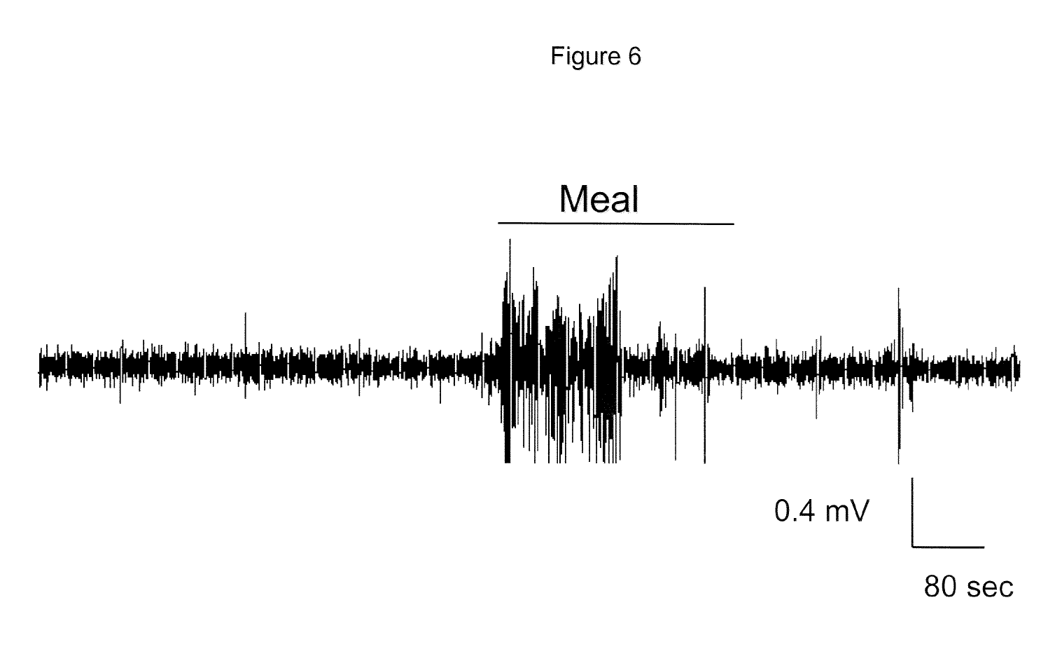

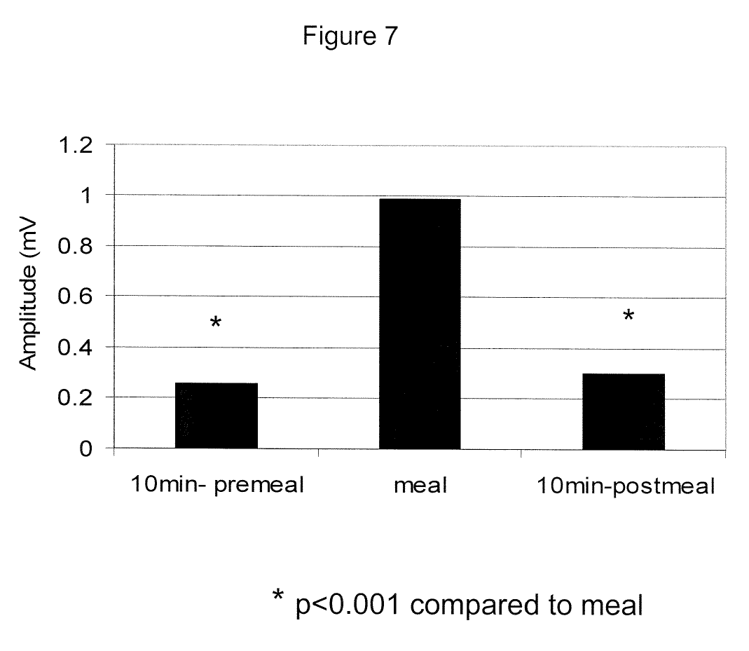

The tracing was visually inspected and then the voltage of the electrical activity recorded for the sphincter was measured. Food intake was characterized by mark increase in the amplitude of electrical activity in the LES that reverted back to baseline value when the meal was terminated (FIGS. 6 and 7). The values are provided in Table 1.

TABLE 1
Data of recording time and amplitudes (in mV)

| | 10 min-pre | meal (4.2 ± 0.9 min) | 10 m-post |
|---|---|---|---|
| Amplitude mV | 0.26 | 0.99 | 0.31 |
| (SD) | 0.1 | 0.23 | 0.1 |

Based on this data, it is possible to reliably detect food intake by tracking the change in the amplitude of electrical activity from electrodes in the LES. From the data, a 3-4 fold increase in amplitude detects food intake. The exact increase that will be considered to indicate food intake may vary depending on a variety of factors. Individual determination for each individual subject may be made.

### Example 4

Four female mongrel dogs underwent an esophagostomy that allowed the introduction of a sleeve manometry catheter into the esophagus. A bipolar electrode was implanted along the longitudinal axis of the LES and connected to an implantable recording device. After recovery, the dogs underwent two tests: (1) telemetric recordings of LES electrical activity, 1 hour fasting and 1 hour postprandial (400 g of canned dog food); and (2) combined recordings of LES electrical recording and esophageal manometry to test the effect of dry swallows, wet swallows and also solid food swallows on LES electrical activity as follows: 20-60 min for spontaneous dry swallows, then 8-12 swallows of 5-10 mil of water and 5-15 min before and after the ingestion of 400 g of canned dog food (FIGS. 6 and 7).

### Example 5

All amplitudes are in mV, mean±SD, ANOVA p<0.05.

The results of Test 1 are as follows. There was a dramatic and characteristic increase in amplitude of LES activity during feeding—amplitude of 0.26±0.1, fasting vs. 0.99±0.23 meal vs. 0.30±0.1, post-prandial, p<0.001.

The results of Test 2 are as follows. LES electrical activity was also related to the type of substance being swallowed: 0.314±0.06, baseline vs. 0.60±.0.11, dry swallows vs. 0.67±0.12, wet swallows vs. 1.06±0.17, solid meal swallows p<0.001 (FIG. 5).

Although the present invention and its advantages have been described in detail, it should be understood that various changes, substitutions and alterations can be made herein without departing from the spirit and scope of the invention. Moreover, the scope of the present application is not intended to be limited to the particular embodiments of the process, manufacture, composition of matter, means, methods and/or steps described in the specification. As one of ordinary skill in the art will readily appreciate from the disclosure of the present invention, processes, manufacture, compositions of matter, means, methods, or steps, presently existing or later to be developed that perform substantially the same function or achieve substantially the same result as the corresponding embodiments described herein may be utilized according to the present invention. Accordingly, the invention is intended to include within its scope such processes, manufacture, compositions of matter, means, methods, or steps.

### REFERENCES

The following references are specifically incorporated herein by reference:

Bohdjalian, A, Prager, G., Aviv, R., Policker, S., Schindler, K., Kretschmer, S. Reiner, R., Zacherl, J., Ludvik, B., Obes. Surg. 16: 627-634, 2004.

Cigaina, V., Gastric pacing as therapy for morbid obesity: Preliminary results, Obes. Surg. 1: S12-S16, 2002.

Cigaina, V., Long-term follow-up of gastric stimulation for obesity: the Mestre 8-year experience, Obes. Surg. 14: S14-S22, 2004.

Favretti, F., De Luca, M., Segato, G., Busetto, L., Ceoloni, A., Magon, A. m Enzi, G., Treatment of morbid obesity with a Transcend Implantable Gastric Stimulator (IGS): A prospective survey, Obes. Surg. 14: 666-670, 2004.

Shikora, S. A., Implantable gastric stimulation for the treatment of severe obesity, Obes. Surg. 14: 545-548, 2004.

What is claimed is:

1. A method of detecting food or drink intake in a subject, comprising:
a) placing one or more electrodes in contact with or proximate to the subject's lower esophageal sphincter (LES); and
b) identifying food or drink intake by monitoring electrical activity in or proximate to the LES using the one or more electrodes.

2. The method of claim 1, wherein two electrodes are placed in contact with or proximate to the subject's LES.

3. The method of claim 1, wherein at least one electrode is placed on one side of the subject's gastro-esophageal junction.

4. The method of claim 1, wherein an increase in the amplitude of LES electrical activity to a value greater than baseline indicates food or drink intake, and a decrease in amplitude from the value greater than baseline to a lower value indicates cessation of food or drink intake.

5. The method of claim 4, wherein an increase in amplitude of more than three fold over baseline indicates food intake.

6. The method of claim 4, wherein an increase in amplitude of about three to about four fold over baseline indicates food intake.

7. The method of claim 4, wherein an increase in amplitude to a value in the range of about 0.55 mV to about 1.57 mV indicates food intake.

8. The method of claim 4, wherein an increase in amplitude to a value in the range of about 0.89 mV to about 1.23 mV indicates food intake.

9. The method of claim 4, wherein an increase in amplitude to a value of about 1.06 mV indicates food intake.

10. The method of claim 4, wherein an increase in amplitude of about two fold over baseline indicates liquid intake.

11. The method of claim 4, wherein an increase in amplitude to a value in the range of about 0.31 mV to about 1.03 mV indicates liquid intake.

12. The method of claim 4, wherein an increase in amplitude to a value in the range of about 0.58 mV to about 0.82 mV indicates liquid intake.

13. The method of claim 4, wherein an increase in amplitude to a value of about 0.7 mV indicates liquid intake.

14. The method of claim 4, wherein a decrease in amplitude of about three to four fold from the value greater than baseline indicates the cessation of food or drink intake.

15. The method of claim 4, wherein a decrease in amplitude to a lower value in the range of about 0.135 mV to about 0.495 mV indicates the cessation of food or drink intake.

16. The method of claim 4, wherein a decrease in amplitude to a lower value in the range of about 0.255 mV to about 0.375 mV indicates the cessation of food or drink intake.

17. The method of claim 4, wherein a decrease in amplitude to a lower value of about 0.315 mV indicates the cessation of food or drink intake.

18. The method of claim 1, further comprising sending a signal indicating start of food or drink intake, ongoing food or drink intake, cessation of food or drink intake, or prior food or drink intake, or any combination thereof, by the subject.

19. The method of claim 18, wherein the signal is sent to a device or system associated with food or drink intake, or cessation of food or drink intake.

20. A method of detecting food or drink intake in a subject, comprising:
a) placing one or more electrodes in contact with or proximate to the subject's lower esophageal sphincter (LES); and
b) identifying food or drink intake by monitoring electrical activity in or proximate to the LES using the one or more electrodes;
wherein an increase in the amplitude of LES electrical activity to a value greater than baseline indicates food or drink intake, and a decrease in amplitude from the value greater than baseline to a lower value indicates cessation of food or drink intake.

21. A device for automatically detecting food or drink intake of a subject, comprising:
a) one or more electrodes, for monitoring electrical activity of the subject's lower esophageal sphincter (LES) or a region proximate to the LES; and
b) a pulse generator, for generating electrical signals based on the monitored electrical activity;
wherein the device is configured to identify food or drink intake by monitoring electrical activity of the LES or the region proximate to the LES using the one or more electrodes.

22. The device of claim 21, further comprising a recording module, for recording electrical data based on the monitored electrical activity.

23. The device of claim 22, further comprising a computer for receiving the electrical signals generated by the pulse generator, and for sending a signal relating to food or drink intake by the subject to another computer or device.

24. The device of claim 22, wherein the recording module comprises a wand and a data logger for recording electrical activity by telemetry.

25. The device of claim 22, wherein the device is configured to send a signal indicating start of food or drink intake, ongoing food or drink intake, cessation of food or drink intake, or prior food or drink intake, or any combination thereof, by the subject.

26. A system for automatically detecting food or drink intake of a subject, comprising
a) one or more electrodes, for monitoring electrical activity of the subject's lower esophageal sphincter (LES) or a region proximate to the LES;
b) a pulse generator, for generating electrical signals relating to the monitored electrical activity; and
c) a computer for interpreting the monitored electrical activity, recording the monitored electrical activity, or both;
wherein the device is configured to identify food or drink intake by monitoring electrical activity of the LES or the region proximate to the LES using the one or more electrodes.

27. The system of claim 26, further comprising a recording device for recording electrical data based on the monitored electrical activity.

28. The system of claim 26, wherein the system is configured to send a signal indicating start of food or drink intake, ongoing food or drink intake, cessation of food or drink intake, or prior food or drink intake, or any combination thereof, by the subject.
